(19) United States
(12) Patent Application Publication
(10) Pub. No.: US 2017/0204023 A1
(43) Pub. Date: Jul. 20, 2017

(54) CATALYST COMPOSITION, AND METHOD FOR PREPARING ALPHA-OLEFIN

(71) Applicant: LOTTE CHEMICAL CORPORATION, Seoul (KR)

(72) Inventors: Tae-jin KIM, Daejeon (KR); Min-jae SHIN, Daejeon (KR); Seung-woong YOON, Daejeon (KR)

(73) Assignee: LOTTE CHEMICAL CORPORATION, Seoul (KR)

(21) Appl. No.: 15/326,666

(22) PCT Filed: Jul. 18, 2014
(86) PCT No.: PCT/KR2014/006553

§ 371 Date: Jan. 17, 2017

(51) Int. Cl. C07C 2/32; B01J 31/04; B01J 31/22

(52) U.S. Cl. CPC C07C 2/32; B01J 31/2295; B01J 31/04; C07C 2531/22; C07C 2531/14; C07C 2531/02; B01J 2231/20; B01J 2531/62; B01J 2531/004

(57) ABSTRACT

The present disclosure relates to a catalyst composition including an organic ligand compound of a specific chemical structure; and a chromium compound and a method for synthesizing alpha-olefin using the catalyst composition, and when the catalyst composition is used, alpha-olefin may be stably synthesized with high selectivity and reaction activity.

### TECHNICAL FIELD

The present disclosure relates to a catalyst composition and a method for preparing alpha-olefin, and more particularly, to a catalyst composition which may stably synthesize alpha-olefin with high selectivity and reaction activity, and a method for preparing alpha-olefin using the catalyst composition.

### BACKGROUND ART

In order to synthesize 1-hexene or 1-octene in the related art, there has been usually used a method for preparing various 1-olefine mixtures by means of oligomerization of ethylene, and then selectively separating and purifying 1-hexene or 1-octene to be targeted, or a method for preparing a 1-olefine mixture using a synthetic gas prepared from coal, and extracting and separating 1-hexene or 1-octene from the 1-olefin mixture, or the like.

However, according to the methods known in the related art, additional separation and purification processes are required because various olefins are simultaneously prepared in addition to commercially useful 1-hexene or 1-octene, and there is a problem in that due to the costs required for the separation and purification, costs of a final product are increased, and the economic efficiency is reduced.

Thus, catalyst technologies and preparation technologies, which may selectively prepare 1-hexene and 1-octene, have been developed, and various studies have been conducted up to now.

For example, methods for oligomerization of ethylene using chromium compounds have been known, and documents, such as U.S. Pat. Nos. 6,943,224, 6,924,248, and 6,900,152, have introduced methods for oligomerization of ethylene through a coordination complex of a transition metal including chromium. Further, U.S. Pat. No. 6,344,594 discloses TaCl₅ as a catalyst for preparing 1-hexene or 1-octene.

However, commercialized catalysts known up to now have low selectivity of 1-hexene and 1-octene, or need to use a specific ligand or compound, and thus have a limitation in that the production costs are high, or the reaction stability of the catalyst is not very high.

### DISCLOSURE

### Technical Problem

The present disclosure has been made in an effort to provide a catalyst composition which may stably synthesize alpha-olefin with high selectivity and reaction activity.

The present disclosure has also been made in an effort to provide a method for synthesizing alpha-olefin using the catalyst composition.

### Technical Solution

The present disclosure provides a catalyst composition including: an organic ligand compound selected from the group consisting of a compound represented by the following Formula 1 and a compound represented by the following Formula 2; and a transition metal compound including a transition metal of Groups 4 to 12.

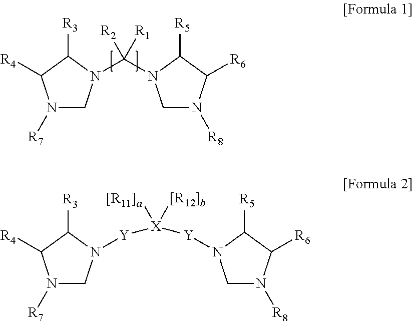

In Formulae 1 and 2,

R₁ to R₈ may be the same as or different from each other, and are each independently selected from a group consisting of hydrogen, a straight-chained or branch-chained alkyl having 1 to 10 carbon atoms, a straight-chained or branch-chained alkenyl having 2 to 10 carbon atoms, an aryl having 6 to 20 carbon atoms, an alkylaryl having 7 to 21 carbon atoms, an arylalkyl having 7 to 21 carbon atoms, a cycloalkyl having 3 to 10 carbon atoms, an alkoxy having 1 to 10 carbon atoms, an aryloxy having 6 to 20 carbon atoms, an alkylsilyl having 1 to 10 carbon atoms, an arylsilyl having 6 to 20 carbon atoms, and halogen,

adjacent two or more of R₁ to R₈ may be linked to form a ring,

n is an integer of 1 to 20,

X is one element selected from the group consisting of boron (B), carbon (C), nitrogen (N), oxygen (O), silicon (Si), phosphorus (P), and sulfur (S),

a and b are each 0 or 1,

R₁₁ and R₁₂ may be the same as or different from each other, and are each independently selected from a group consisting of a straight-chained or branch-chained alkyl having 1 to 10 carbon atoms, a straight-chained or branch-chained alkenyl having 2 to 10 carbon atoms, an aryl having 6 to 20 carbon atoms, an alkylaryl having 7 to 21 carbon atoms, an arylalkyl having 7 to 21 carbon atoms, a cycloalkyl having 3 to 10 carbon atoms, an alkoxy having 1 to 10 carbon atoms, an aryloxy having 6 to 20 carbon atoms, an alkylsilyl having 1 to 10 carbon atoms, an arylsilyl having 6 to 20 carbon atoms, and halogen,

R₁₁ and R₁₂ may be linked to form a ring, and

Y and Z may be the same as or different from each other, and are each independently selected from a group consisting of a straight-chained or branch-chained alkylene group having 1 to 20 carbon atoms, a straight-chained or branch-chained alkenylene group having 2 to 20 carbon atoms, an arylene group having 6 to 20 carbon atoms, an alkylarylene group having 7 to 20 carbon atoms, and an arylalkylene group having 7 to 20 carbon atoms.

Further, the present disclosure provides a method for preparing alpha-olefin using the catalyst composition.

### Advantageous Effects

According to the present disclosure, it is possible to provide a catalyst composition which may stably synthesize alpha-olefin with high selectivity and reaction activity, and a method for synthesizing alpha-olefin using the catalyst composition. In particular, when the catalyst composition is used, 1-hexene and 1-octene may be stably synthesized while securing high selectivity and catalytic activity.

### BEST MODE

Hereinafter, a catalyst composition according to a specific exemplary embodiment and a method for preparing alpha-olefin will be described in more detail.

In the present specification, alkyl means a monovalent functional group derived from alkane, and alkenyl means a monovalent functional group derived from alkene.

Further, aryl means a monovalent functional group derived from arene, an alkylaryl group means an aryl group into which one or more straight-chained or branch-chained alkyl groups are introduced, and an arylalkyl group means a straight-chained or branch-chained alkyl group into which one or more aryl groups are introduced.

In addition, cycloalkyl means a monovalent functional group derived from cycloalkane, alkoxy means a monovalent functional group in which a straight-chained or branch-chained alkyl group is bonded to oxygen, and aryloxy means a monovalent functional group in which an aryl group is bonded to oxygen.

Furthermore, alkylsilyl and arylsilyl mean a silyl group to which an alkyl group is bonded and a silyl group to which an aryl group is bonded, respectively.

Further, alkylene means a divalent functional group derived from alkane, alkenylene means a divalent functional group derived from alkene, arylene means a divalent functional group derived from arene, an alkylarylene group means an arylene group in which one or more alkyl groups are substituted, and arylalkylene means an alkyene group in which one or more aryl groups are substituted.

An exemplary embodiment of the present disclosure provides a catalyst composition including: an organic ligand compound selected from the group consisting of the compound represented by Formula 1 and the compound represented by Formula 2; and a transition metal compound including a transition metal of Groups 4 to 12.

The present inventors newly synthesized the compound represented by Formula 1 and the compound represented by Formula 2, and confirmed through an experiment that when these compounds are used together with a transition metal compound, alpha-olefin could be stably synthesized from ethylene with high selectivity and reaction activity, thereby completing the invention.

In particular, when the compound of Formula 1, the compound of Formula 2, or a mixture thereof is used together with a transition metal compound, 1-hexene and 1-octene may be efficiently and stably synthesized with high selectivity while securing improved catalytic activity as compared to other transition metal catalysts previously known.

As described above, the catalyst composition according to an exemplary embodiment may be used in a reaction of synthesizing alpha-olefin from ethylene. The alpha-olefin to be synthesized may include 1-hexene and 1-octene, and may additionally include other alpha-olefins.

The specific contents of Formula 1 are the same as those described above, and more specifically, R₁, R₂, R₃, R₄, R₅, and R₆ of Formula 1 may be the same as or different from each other, and may be each hydrogen, a straight-chained or branch-chained alkyl group having 1 to 10 carbon atoms, a cycloalkyl group having 4 to 8 carbon atoms, or an aryl group having 6 to 20 carbon atoms.

In addition, R₇ and R₈ of Formula 1 may be the same as or different from each other, and may be each a straight-chained or branch-chained alkyl group having 1 to 10 carbon atoms, a cycloalkyl group having 4 to 8 carbon atoms, an aryl group having 6 to 20 carbon atoms, an alkylaryl group having 7 to 20 carbon atoms, or an arylalkyl group having 7 to 20 carbon atoms.

Furthermore, the specific contents of Formula 2 are the same as those described above, and more specifically, R₃, R₄, R₅, and R₆ of Formula 2 may be the same as or different from each other, and may be each hydrogen, a straight-chained or branch-chained alkyl group having 1 to 10 carbon atoms, a cycloalkyl group having 4 to 8 carbon atoms, or an aryl group having 6 to 20 carbon atoms.

R₇ and R₈ of Formula 2 may be the same as or different from each other, and may be each a straight-chained or branch-chained alkyl group having 1 to 10 carbon atoms, a cycloalkyl group having 4 to 8 carbon atoms, an aryl group having 6 to 20 carbon atoms, an alkylaryl group having 7 to 20 carbon atoms, or an arylalkyl group having 7 to 20 carbon atoms.

X of Formula 2 may be nitrogen, Y and Z may be the same as or different from each other, and may be each an alkylene group having 1 to 5 carbon atoms, and a may be 1 and b may be 0.

Further, R₁₁ of Formula 2 may be a straight-chained or branch-chained alkyl group having 1 to 10 carbon atoms, a cycloalkyl group having 4 to 8 carbon atoms, an aryl group having 6 to 20 carbon atoms, an alkylaryl group having 7 to 20 carbon atoms, or an arylalkyl group having 7 to 20 carbon atoms.

In the catalyst composition of an exemplary embodiment, the organic ligand compound and the transition metal compound may form a coordination bond, and specifically, an unshared electron pair of the compound of Formula 1 or the compound of Formula 2 and the transition metal of the transition metal compound may form a coordination bond.

The transition metal compound may include a transition metal of Groups 4 to 12, and specifically, may be the transition metal itself or an organic compound including the transition metal.

The transition metal compound may include a chromium compound.

The chromium compound may be a chromium metal or an organic compound including chromium. Specifically, the chromium compound may be chromium, chromium (III) acetylacetonate, tris-tetrahydrofuran chromium trichloride, chromium (III) 2-ethylhexanoate, or a mixture of two or more thereof.

Meanwhile, the catalyst composition may include the organic ligand compound in an amount of 0.5 mole to 2.0 mole, preferably 0.8 mole to 1.2 mole, based on 1 mole of the transition metal compound.

When the content of the organic ligand compound is too low compared to the transition metal compound, the reaction active site of the catalyst composition may not be formed, or the activity of the catalyst may deteriorate, and the selectivity for alpha-olefin to be synthesized, and the like may deteriorate. In addition, when the content of the organic ligand compound is too high compared to the transition metal compound, the reaction active site of the catalyst composition may not be sufficiently exposed to the outside, and accordingly, the activity of the catalyst or the selectivity for alpha-olefin to be synthesized, and the like may rather deteriorate.

Meanwhile, the catalyst composition may further include a co-catalyst. Moreover, specific examples of the co-catalyst include a compound represented by the following Formula 11, a compound represented by the following Formula 12, a compound represented by the following Formula 13, or a mixture of two or more thereof.

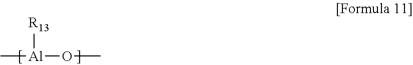

In Formula 11, R₁₃ may be an alkyl group having 1 to 10 carbon atoms, and r may be an integer of 1 to 70,

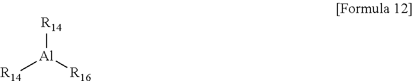

In Formula 12, R₁₄, R₁₅, and R₁₆ may be the same as or different from each other, and may be each an alkyl group having 1 to 10 carbon atoms, an alkenyl group having 2 to 10 carbon atoms, an alkoxy group having 1 to 10 carbon atoms, or halogen, and at least one of R₁₄, R₁₅, and R₁₆ may be an alkyl group having 1 to 10 carbon atoms,

[L-H]⁺[Z(E)₄]⁻ or [L]⁺[Z(E)₄]⁻, [Formula 13]

In Formula 11, L is a neutral or cationic Lewis base, [L-H]⁺ or [L]⁺ is a Bronsted acid, H is a hydrogen atom, and Z is a Group 13 element, and

E's are optionally the same as or different from each other, and are each independently an aryl group having 6 to 20 carbon atoms or an alkyl group having 1 to 20 carbon atoms, wherein the aryl group and the alkyl group of E are each independently are substituted or unsubstituted with one or more functional groups selected from a group consisting of halogen, a hydrocarbyl having 1 to 20 carbon atoms, an alkoxy functional group having 1 to 20 carbon atoms, and a phenoxy functional group.

The compound of Formula 11 or 12 may serve as a scavenger to remove impurities acting as a poison to a catalyst in reactants, or may serve to cationize or activate a central metal of a transition metal compound to allow ethylene to react well with the central metal.

In the catalyst composition, the mole number of the transition metal compound: the mole number of the compound of Formula 11 or 12 may be 1:1 to 1:5,000, preferably 1:10 to 1:1,000, and more preferably 1:20 to 1:500. When the molar ratio is less than 1:1, the effect of adding the co-catalyst is minimal, and when the molar ratio is more than 1:5,000, an excess amount of an alkyl group and the like, which fail to participate in the reaction and remain, may rather suppress the catalyst reaction to serve as a catalyst poison, and accordingly, there may occur a problem in that an excess amount of aluminum or boron remains in a polymer because side reactions proceed.

The compound of Formula 11 may have a chain, cyclic or network structure, and specific examples thereof include methylaluminoxane, ethylaluminoxane, butylaluminoxane, hexylaluminoxane, octylaluminoxane, decylaluminoxane, and the like.

Specific examples of the compound of Formula 12 include: trialkylaluminum such as trimethylaluminum, triethylaluminum, tributylaluminum, trihexylaluminum, trioctylaluminum, and tridecyl aluminum; dialkylaluminum alkoxide such as dimethylaluminum methoxide, diethylaluminum methoxide, and dibutylaluminum methoxide; dialkylaluminum halide such as dimethylaluminum chloride, diethylaluminum chloride, and dibutylaluminum chloride; alkylaluminum dialkoxide such as methylaluminum dimethoxide, ethylaluminum dimethoxide, and butylaluminum dimethoxide; and alkylaluminum dihalide such as methylaluminum dichloride, ethylaluminum dichloride, and butyl aluminum dichloride.

The compound of Formula 13 may serve to cationize or activate the central metal of the transition metal compound to allow ethylene to react well with the central metal, and may include a non-coordination bonding anion compatible with a cation being Bronsted acid. A preferred anion has a relatively large size and contains a single coordination bonding complex compound including a metalloid. In particular, a compound containing a single boron atom in an anion portion is widely used. From the viewpoint, a salt containing an anion including a coordination bonding complex compound containing a single boron atom is preferred.

In the catalyst composition, the mole number of the transition metal compound: the mole number of the compound of Formula 13 may be 1:1 to 1:10, preferably 1:1 to 1:4. When the molar ratio is less than 1:1, the degree of activity of the transition metal catalyst may not be sufficient because the metal compound is not completely activated due to the relatively small amount of the co-catalyst, and when the molar ratio is more than 1:10, the degree of activity of the transition metal catalyst may be increased, but there may occur a problem in that production costs are significantly increased because co-catalysts are used more than needed.

Specific examples of the compound of Formula 13 include trimethylammonium tetraphenylborate, triethylammonium tetraphenylborate, tripropyl ammonium tetraphenylborate, tributylammonium tetraphenylborate, trimethylammonium tetrakis(pentafluorophenyl)borate, triethylammonium tetraki s(pentafluorophenyl)borate, tripropylammonium tetrakis(pentafluorophenyl)borate, tributylammonium tetrakis(pentafluorophenyl)borate, anilinium tetraphenylborate, anilinium tetrakis(pentafluorophenyl)borate, pyridinium tetraphenylborate, pyridinium tetrakis(pentafluorophenyl)borate, ferrocenium tetrakis(pentafluorophenyl)borate, silver tetraphenylborate, silver tetrakis(pentafluorophenyl) borate, tris(pentafluorophenyl)borane, tris(2,3,5,6-tetrafluorophenyl)borane, tris(2,3,4,5-tetraphenylphenyl)borane, and tris(3,4,5-trifluorophenyl)borane.

Meanwhile, according to another exemplary embodiment of the present disclosure, a method for preparing alpha-olefin using the above-described catalyst composition may be provided.

As described above, when using a catalyst composition including: an organic ligand compound selected from the group consisting of the compound of Formula 1 and the compound of Formula 2; and a transition metal compound including a transition metal of Groups 4 to 12, alpha-olefin may be stably synthesized from ethylene with high selectivity and reaction activity. In particular, when the catalyst composition is used, 1-hexene and 1-octene may be efficiently and stably synthesized with high selectivity while securing improved catalytic activity as compared to other transition metal catalysts previously known.

Specific contents of the compound of Formula 1, the compound of Formula 2, and the transition metal compound are the same as those described above.

As described above, the catalyst composition may selectively further include a co-catalyst.

In the method for preparing alpha-olefin according to an exemplary embodiment, alpha-olefin may be synthesized by reacting the catalyst composition with ethylene in an organic solvent. Specifically, alpha-olefin may be synthesized by introducing the catalyst composition, ethylene, and the organic solvent into a reactor to perform the reaction and oligomerization of ethylene, and particularly, 1-hexene and 1-octene may be prepared by subjecting ethylene to trimerization or tetramerization reaction.

The organic solvent which may be used is not particularly limited, but for example, a purified hydrocarbon compound (for example, n-hexane and n-heptane, cyclohexane, toluene, benzene, and the like) may be used.

In the method for preparing alpha-olefin according to an exemplary embodiment, the catalyst composition and the organic solvent may be simultaneously or sequentially injected into a reactor and mixed, and the transition metal compound and the organic ligand compound are first reacted to prepare a complex, and then a reaction may also be performed by introducing a catalyst solution, which is prepared by reacting a co-catalyst with the solvent, into a reactor.

In the method for preparing alpha-olefin according to an exemplary embodiment, in the presence of the catalyst, the temperature for the trimerization or tetramerization reaction of ethylene is 0° C. to 200° C., preferably 20° C. to 150° C., and the reaction pressure is 1 bar to 150 bar, preferably 20 bar to 100 bar.

The present disclosure will be described in more detail in the following Examples. However, the following Examples are only for exemplifying the present disclosure, and the content of the present disclosure is not limited by the following Examples.

(1) In the following Examples, Comparative Examples, and Experimental Examples, all the synthesis reactions were carried out under an inert atmosphere such as nitrogen or argon, and a standard Schlenk technology and a glove box technology were used.

(2) A solvent for synthesis, such as tetrahydrofuran (THF), n-hexane, n-pentane, diethyl ether, methylene chloride (CH₂Cl₂), and toluene (C₇H₈) was allowed to pass through an activated alumina column to remove moisture, and then was used while being stored on an activated molecular sieve (Molecular Sieve 5 Å, manufactured by Yakuri Pure Chemicals Co., Ltd.), and deuterium-substituted chloroform (Chloroform-d, CDCl₃), deuterated benzene (benzene-d6, C₆D₆), and dimethyl sulfoxide (dimethylsulfoxide-d6, C₂D₆SO) was used for the NMR structural analysis of the compound and were purchased from Cambridge Isotope Laboratories Inc., and then used while being dried on an activated molecular sieve (Molecular Sieve 5 Å, manufactured by Yakuri Pure Chemicals Co., Ltd.).

(3) All the reagents used below, and the like were purchased from Sigma-Aldrich Chemical Co., and used without purification.

(4) 1H NMR was measured by using a Bruker Avance 400 spectrometer at normal temperature, the chemical shifts of the NMR spectrum were indicated based on the chemical shifts δ=7.24 ppm, 7.16 ppm, and 2.50 ppm, which deuterated chloroform (CDCl₃), deuterated benzene (C₆D₆), and dimethyl sulfoxide (C₂D₆SO) indicate, respectively.

### Synthesis Examples: Synthesis of Organic Ligand Compound

### 1. Synthesis Example 1: 1,2-[C₆H₂(CH₃)₃-imidazole]₂-C₂H₄

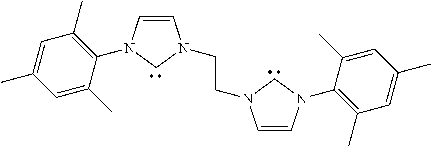

### (1) Synthesis Example 1-1: Synthesis of C₆H₂(CH₃)₃-imidazole

Ammonium chloride (5.35 g, 100 mmol) was dissolved in 20 mL of distilled water, and then a mixed solution of 100 mL of water, 100 mL of 1,4-dioxane, paraformaldehyde (3.00 g, 100 mmol), an aqueous glyoxal solution (11.5 mL, 100 mmol), and a mesityl ammonium salt (13.2 g, 200 mmol) was stirred and slowly added dropwise to the solution. After the dropwise addition was completed, the temperature of the reaction solution was raised to 100° C., and the reaction solution was refluxed for 3 hours.

After the reflux, the temperature was lowered to 0° C., and an aqueous sodium hydroxide solution (1 M) was added dropwise thereto until the pH became 12 or more. After the dropwise addition was completed, the resulting product was extracted with 1,500 mL of hexane, and dried by using anhydrous magnesium sulfate. The solvent was evaporated by a rotary evaporator to obtain 2.71 g of a white solid (S1-1). 1H NMR (CDCl3): δ 7.50 (s), 6.98 (s), 6.92 (s), 2.35 (s), 2.00 (s).

### (2) Synthesis Example 1-2: [1,2-{C₆H₂(CH₃)₃-imidazole}₂-C₂H₄]Br₂

1,2-dibromoethane (0.94 g, 5 mmol) and the solid (S1-1) (1.72 g, 10 mmol) obtained in Synthesis Example 1-1 were dissolved in 100 mL of toluene, and then the resulting solution was stirred. After the stirring was completed, the temperature of the reaction solution was raised to 120° C., and then the reaction solution was refluxed for 18 hours. After the reflux was completed, the temperature of the reaction solution was lowered to normal temperature, the reaction solution was filtered by using a cannula, and then 0.73 g of a white solid (S1-2) was obtained.

1H NMR (DMSO-d6): δ 9.48 (s), 8.12 (s), 7.96 (s), 7.15 (s), 4.29 (t), 3.39 (s), 2.33 (s), 2.01 (s), 1.90 (s), 1.32 (s).

### (3) Synthesis Example 1-3: 1,2-[C₆H₂(CH₃)₃-imidazole]₂-C₂H₄

Potassium bistrimethylsilylamide (1 mL, 1 M THF solution) was slowly added dropwise to the solid (S1-2) (0.72 g, 1 mmol) obtained in Synthesis Example 1-2 at normal temperature. After the dropwise addition was completed, the mixed solution was reacted at normal temperature for 12 hours. After the reaction was completed, the resulting product was filtered and concentrated by using a cannula, and was recrystallized at −50° C. to obtain 0.58 g of a white solid (S1-3).

1H NMR (CDCl3): δ 7.32 (t), 7.21 (d), 7.19 (s), 6.49 (s), 6.29 (s), 1.14 (s)

### 2. Synthesis Example 2: 1,4-[C₆H₂(CH₃)₃-imidazole]₂-C₄H₈

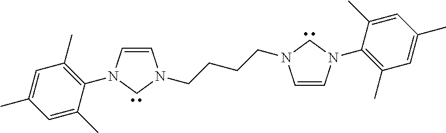

### (1) Synthesis Example 2-1: [1,4-{C₆H₂(CH₃)₃-imidazole}₂-C₄H₈]Br₂

1,4-dibromobutane (1.08 g, 5 mmol) and the solid (S1-1) (1.72 g, 10 mmol) obtained in Synthesis Example 1-1 were dissolved in 100 mL of toluene, and then the resulting solution was stirred.

The temperature of the reaction solution was raised to 120° C., and then the reaction solution was refluxed for 18 hours. After the reflux was completed, the temperature of the reaction solution was lowered to normal temperature, the reaction solution was filtered by using a cannula, and then 0.64 g of a white solid (S2-1) was obtained.

1H NMR (DMSO-d6): δ 9.10 (s), 7.99 (s), 7.21 (s), 7.11 (s), 4.26 (t), 3.09 (s), 2.33 (m), 1.21 (m), 1.09 (m).

### (2) Synthesis Example 2-2: 1,4-[C₆H₂(CH₃)₃-imidazole]₂-C₄H₈

Potassium bistrimethylsilylamide (1 mL, 1 M THF solution) was slowly added dropwise to the solid (S2-1) (0.92 g, 1 mmol) obtained in Synthesis Example 2-1 at normal temperature.

After the dropwise addition was completed, the mixed solution was reacted at normal temperature for 12 hours. After the reaction was completed, the resulting product was filtered and concentrated by using a cannula, and was recrystallized at −50° C. to obtain 0.28 g of a white solid (S2-2).

1H NMR (CDCl3): δ 7.45 (s), 6.37 (s), 6.00 (s), 2.39 (s), 2.21 (s), 1.11 (m).

### 3. Synthesis Example 3: 1,6-[C₆H₂ (CH₃)₃-imidazole]₂-C₆H₁₂

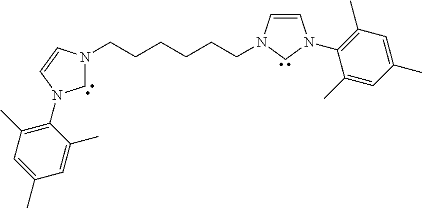

### (1) Synthesis Example 3-1: [1,6-{C₆H₂(CH₃)₃-imidazole}₂-C₆H₁₂]Br₂

1,6-dibromohexane (0.94 g, 5 mmol) and the solid (S1-1) (1.72 g, 10 mmol) obtained in Synthesis Example 1-1 were dissolved in 100 mL of toluene, and then the resulting solution was stirred. The temperature of the reaction solution was raised to 120° C., and then the reaction solution was refluxed for 18 hours. After the reflux was completed, the temperature of the reaction solution was lowered to normal temperature, the reaction solution was filtered by using a cannula, and then 0.92 g of a white solid (S3-1) was obtained.

1H NMR (DMSO-d6): δ 9.12 (s), 8.00 (s), 7.32 (s), 7.21 (s), 4.11 (t), 3.12 (s), 2.21 (m), 2.12 (m), 1.45 (m), 1.11 (m).

### (2) Synthesis Example 3-2: 1,6-[C₆H₂(CH₃)₃-imidazole]₂-C₆H₁₂

Potassium bistrimethylsilylamide (1 mL, 1 M THF solution) was slowly added dropwise to the solid (S3-1) (0.88 g, 1 mmol) obtained in Synthesis Example 3-1 at normal temperature.

After the dropwise addition was completed, the mixed solution was reacted at normal temperature for 12 hours. After the reaction was completed, the resulting product was filtered and concentrated by using a cannula, and was recrystallized at −50° C. to obtain 0.45 g of a white solid (S3-2).

1H NMR (CDCl3): δ 7.32 (s), 6.21 (s), 6.12 (s), 2.61 (s), 2.31 (s), 1.88 (m), 1.23 (m).

### 4. Synthesis Example 4: 1,2-[C₃H₇-imidazole]₂-C₂H₄

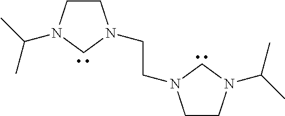 [SMILES: CC(C)N1CCN(CCN2CCN(C(C)C)C2)C1]

### (1) Synthesis Example 4-1: C₃H₇-imidazole

Ammonium chloride (5.35 g, 100 mmol) was dissolved in 20 mL of distilled water, and then a mixed solution of 100 mL of water, 100 mL of 1,4-dioxane, paraformaldehyde (3.00 g, 100 mmol), an aqueous glyoxal solution (11.5 mL, 100 mmol), and an isopropyl ammonium salt (7.55 g, 200 mmol) was stirred and slowly added dropwise to the solution. After the dropwise addition was completed, the temperature of the reaction solution was raised to 100° C., and the reaction solution was refluxed for 3 hours.

After the reflux, the temperature was lowered to 0° C., and an aqueous sodium hydroxide solution (1 M) was added dropwise thereto until the pH became 12 or more. After the dropwise addition was completed, the resulting product was extracted with 1,500 mL of hexane, and dried by using anhydrous magnesium sulfate. The solvent was evaporated by a rotary evaporator to obtain 3.59 g of a white solid (S4-1).

1H NMR (CDCl3): δ 7.11 (s), 2.21 (m), 1.77 (d).

### (2) Synthesis Example 4-2: [1,2-{C₃H₇-imidazole}₂-C₂H₄]Br₂

1,4-dibromobutane (1.08 g, 5 mmol) and the solid (S4-1) (1.72 g, 10 mmol) obtained in Synthesis Example 4-1 were dissolved in 100 mL of toluene, and then the resulting solution was stirred. The temperature of the mixed solution was raised to 120° C., and then the reaction solution was refluxed for 18 hours. After the reaction, the temperature was lowered to normal temperature, the resulting product was filtered by using a cannula, and then 0.65 g of a white solid (S4-2) was obtained.

1H NMR (DMSO-d6): δ 10.20 (s), 9.22 (s), 4.12 (t), 3.66 (s), 2.33 (m), 1.11 (d).

### (3) Synthesis Example 4-3: 1,2-[C₃H₇-imidazole]₂-C₂H₄

Potassium bistrimethylsilylamide (1 mL, 1 M THF solution) was slowly added dropwise to the solid (S4-2) (0.87 g, 1 mmol) obtained in Synthesis Example 4-2 at normal temperature.

After the dropwise addition was completed, the mixed solution was reacted at normal temperature for 12 hours. After the reaction was completed, the resulting product was filtered and concentrated by using a cannula, and was recrystallized at −50° C. to obtain 0.33 g of a white solid (S4-3).

1H NMR (CDCl3): δ 7.21 (s), 6.32 (s), 2.32 (m), 2.23 (m), 1.12 (m).

### 5. Synthesis Example 5: [{C₆H₂(CH₃₃-imidazole}-(C₂H₄)]₂—C₃H₇N

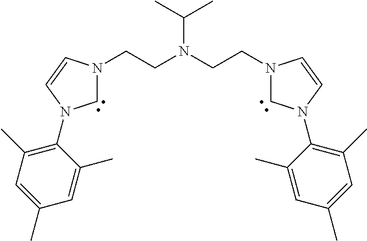

### (1) Synthesis Example 5-1: Br₂[{₆H₂(CH₃)₃-imidazole}-(C₂H₄)]₂—C₃H₇N

(BrC₂H₄)₂—C₃H₇N (1.21 g, 5 mmol) and the solid (S1-1) (1.72 g, 10 mmol) obtained in Synthesis Example 1-1 were dissolved in 100 mL of toluene, and then the resulting solution was stirred. The temperature of the mixed solution was raised to 120° C., and then the reaction solution was refluxed for 18 hours. After the reaction, the temperature was lowered to normal temperature, the resulting product was filtered by using a cannula, and then 0.42 g of a white solid (S5-1) was obtained.

1H NMR (DMSO-d6): δ 9.22 (s), 8.12 (s), 7.34 (s), 7.12 (s), 4.33 (t), 4.22 (s), 3.09 (b), 2.11 (m), 1.32 (m), 1.08 (m).

### (2) Synthesis Example 5-2: [{C₆H₂(CH₃)₃-imidazole}-(C₂H₄)]₂—C₃H₇N

Potassium bistrimethylsilylamide (1 mL, 1 M THF solution) was slowly added dropwise to the solid (S5-1) (0.92 g, 1 mmol) obtained in Synthesis Example 5-1 at normal temperature. After the dropwise addition was completed, the mixed solution was reacted at normal temperature for 12 hours. After the reaction was completed, the resulting product was filtered and concentrated by using a cannula, and was recrystallized at −50° C. to obtain 0.47 g of a white solid (S5-2).

1H NMR (DMSO-d6): δ 8.12 (s), 7.22 (s), 6.93 (s), 6.77 (s), 5.23 (t), 4.99 (s), 2.23 (m), 1.12 (m), 0.92 (m).

### 6. Synthesis Example 6: [{C₃H₇-imidazole}-(C₂H₄)]₂—C₃H₇N

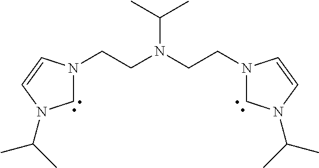 [SMILES: CC(C)N1C=CN(CCN(CCN2C=CN(C(C)C)C2)C(C)C)C1]

### (1) Synthesis Example 6-1: Br₂[{C₃H₇-imidazole}-(C₂H₄)]₂—C₃H₇N

(BrC₂H₄)₂—C₃H₇N (1.91 g, 5 mmol) and the solid (S1-1) (1.72 g, 10 mmol) obtained in Synthesis Example 1-1 were dissolved in 100 mL of toluene, and then the resulting solution was stirred. The temperature of the mixed solution was raised to 120° C., and then the reaction solution was refluxed for 18 hours. After the reaction, the temperature was lowered to normal temperature, the resulting product was filtered by using a cannula, and then 0.97 g of a white solid (S6-1) was obtained.

1H NMR (DMSO-d6): δ 8.09 (s), 7.43 (s), 7.12 (s), 6.89 (s), 6.21 (t), 5.33 (s), 2.11 (m), 1.53 (m), 1.44 (m).

### (2) Synthesis Example 6-2: [{C₃H₇-imidazole}-(C₂H₄)]₂—C₃H₇N

Potassium bistrimethylsilylamide (1 mL, 1 M THF solution) was slowly added dropwise to the solid (S6-1) (0.9 g, 1 mmol) obtained in Synthesis Example 6-1 at normal temperature. After the dropwise addition was completed, the mixed solution was reacted at normal temperature for 12 hours. After the reaction was completed, the resulting product was filtered and concentrated by using a cannula, and was recrystallized at −50° C. to obtain 0.47 g of a white solid (S6-2).

1H NMR (DMSO-d6): 3.99 (t), 1.59 (m), 1.21 (m).

### 7. Synthesis Example 7: 1,2-[CH₃-imidazole]₂-C₂H₄

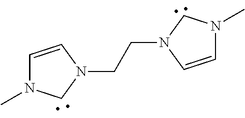

### (1) Synthesis Example 7-1: CH₃-imidazole

Ammonium chloride (5.35 g, 100 mmol) was dissolved in 20 mL of distilled water, and then a mixed solution of 100 mL of water, 100 mL of 1,4-dioxane, paraformaldehyde (3.00 g, 100 mmol), an aqueous glyoxal solution (11.5 mL, 100 mmol), and a methyl ammonium salt (3.4 g, 200 mmol) was vigorously stirred and slowly added dropwise to the solution. After the dropwise addition was completed, the temperature of the reaction solution was raised to 100° C., and the reaction solution was refluxed for 3 hours.

After the reflux, the temperature was lowered to 0° C., and an aqueous sodium hydroxide solution (1 M) was added dropwise thereto until the pH became 12 or more. After the dropwise addition was completed, the resulting product was extracted with 1,500 mL of hexane, and dried by using anhydrous magnesium sulfate. The solvent was evaporated by a rotary evaporator to obtain 5.74 g of a white solid (S7-1).

1H NMR (CDCl3): δ 7.34 (s), 7.11 (s), 0.91 (s).

### (2) Synthesis Example 7-2: [1,2-{CH₃-imidazole}₂-C₂H₄]Br₂

1,2-dibromoethane (0.94 g, 5 mmol) and the solid (S7-1; 0.82 g, 10 mmol) obtained in Synthesis Example 7-1 were dissolved in 100 mL of toluene, and then the resulting solution was stirred. The temperature of the mixed solution was raised to 120° C., and then the reaction solution was refluxed for 18 hours. After the reaction, the temperature was lowered to normal temperature, the resulting product was filtered by using a cannula, and then 0.70 g of a white solid (S7-2) was obtained.

1H NMR (DMSO-d6): δ 9.32 (s), 7.23 (s), 7.13 (s), 7.01 (m) 4.21 (t), 3.24 (s), 2.44 (s), 2.02 (s), 1.25 (s).

### (3) Synthesis Example 7-3: 1,2-[CH₃-imidazole]₂-C₂H₄

Potassium bistrimethylsilylamide (1 mL, 1 M THF solution) was slowly added dropwise to the solid (S7-2; 0.35 g, 1 mmol) obtained in Synthesis Example 7-2 at normal temperature.

After the dropwise addition was completed, the mixed solution was reacted at normal temperature for 12 hours. After the reaction was completed, the resulting product was filtered and concentrated by using a cannula, and was recrystallized at −50° C. to obtain 0.12 g of a white solid (S7-3).

1H NMR (CDCl3): δ 7.53 (s), 6.21 (s), 2.66 (m), 2.32 (m), 1.11 (m).

### 8. Synthesis Example 8: 1,2-[C₆H₅-imidazole]₂-C₂H₄

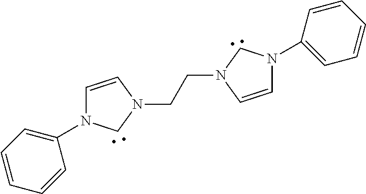 [SMILES: C1=CN(c2ccccc2)CN1CCN1C=CN(c2ccccc2)C1]

### (1) Synthesis Example 8-1: C₆H₅-imidazole

Ammonium chloride (5.35 g, 100 mmol) was dissolved in 20 mL of distilled water, and then a mixed solution of 100 mL of water, 100 mL of 1,4-dioxane, paraformaldehyde (3.00 g, 100 mmol), an aqueous glyoxal solution (11.5 mL, 100 mmol), and a phenyl ammonium salt (9.45 g, 200 mmol) was vigorously stirred and slowly added dropwise to the solution. After the dropwise addition was completed, the temperature of the reaction solution was raised to 100° C., and the reaction solution was refluxed for 3 hours.

After the reflux, the temperature was lowered to 0° C., and an aqueous sodium hydroxide solution (1 M) was added dropwise thereto until the pH became 12 or more. After the dropwise addition was completed, the resulting product was extracted with 1,500 mL of hexane, and dried by using anhydrous magnesium sulfate. The solvent was evaporated by a rotary evaporator to obtain 3.41 g of a white solid (S8-1).

1H NMR (CDCl3): δ 7.56 (s), 7.07 (s), 2.14 (m), 1.01 (s).

### (2) Synthesis Example 8-2: [1,2-{C₆H₅-imidazole}₂-C₂H₄]Br₂

1,2-dibromoethane (0.94 g, 5 mmol) and the solid (S8-1; 1.94 g, 10 mmol) obtained in Synthesis Example 8-1 were dissolved in 100 mL of toluene, and then the resulting solution was stirred. The temperature of the mixed solution was raised to 120° C., and then the reaction solution was refluxed for 18 hours. After the reaction, the temperature was lowered to normal temperature, the resulting product was filtered by using a cannula, and then 0.94 g of a white solid (S8-2) was obtained.

1H NMR (DMSO-d6): δ 9.76 (s), 7.53 (s), 7.34 (s), 7.05 (m) 4.52 (t), 3.44 (s), 2.11 (s), 1.94 (s), 1.21 (s).

### (3) Synthesis Example 8-3: 1,2-[C₆H₅-imidazole]₂-C₂H₄

Potassium bistrimethylsilylamide (1 mL, 1 M THF solution) was slowly added dropwise to the solid (S8-2; 0.48 g, 1 mmol) obtained in Synthesis Example 8-2 at normal temperature.

After the dropwise addition was completed, the mixed solution was reacted at normal temperature for 12 hours. After the reaction was completed, the resulting product was filtered and concentrated by using a cannula, and was recrystallized at −50° C. to obtain 0.16 g of a white solid (S8-3).

1H NMR (CDCl3): δ 7.78 (s), 7.37 (m), 6.55 (s), 2.25 (m), 1.92 (m), 1.02 (m).

### Example 1: Oligomerization Reaction of Ethylene Using Tris-Tetrahydrofuran Chromium Trichloride

A 2 L stainless steel reactor was filled with nitrogen, and then 1 L of cyclohexane was added thereto, 9.0 mmol (10 wt % in toluene, Albermale) of MAO was added thereto, and then the temperature was increased to 45° C. 11 mg (0.030 mmol) of tris-tetrahydrofuran chromium trichloride in 10 mL of toluene was placed in a 50 ml Schlenk container in a glove box, and was mixed with 0.030 mmol of the ligand each obtained in Synthesis Example 1, and the resulting mixture was stirred at normal temperature for 5 minutes, and then was added to the reactor.

A pressure reactor was filled with ethylene at 30 bar, and the ethylene was stirred at a stirring rate of 300 rpm. After 1 hour, ethylene was stopped to be supplied to the reactor, the stirring was stopped to terminate the reaction, and the reactor was cooled to less than 10° C.

An excess amount of ethylene in the reactor was released, and then ethanol mixed with 10 vol % hydrochloric acid was injected into the liquid contained in the reactor. A small amount of an organic layer sample was allowed to pass through the silica gel and dried, and then analyzed by GC-FID. The solid wax/polymer products were separated by filtering the other organic layer. These solid products were dried in an oven at 80° C. for 8 hours, and then the weights were measured to obtain polyethylene.

### Examples 2 to 8: Oligomerization Reaction of Ethylene Using Tris-Tetrahydrofuran Chromium Trichloride

The organic layer samples and polyethylene were obtained in the same manner as in Example 1, except that the ligands obtained in Synthesis Examples 2 to 8 were each used instead of the ligand obtained in Synthesis Example 1.

### Example 9: Oligomerization Reaction of Ethylene Using Chromium (III) 2-Ethylhexanoate

The organic layer sample and polyethylene were obtained in the same manner as in Example 1, except that 14 mg (0.03 mmol) of chromium (III) 2-ethylhexanoate was placed instead of 11 mg (0.030 mmol) of tris-tetrahydrofuran chromium trichloride in 10 mL of toluene.

### Example 10: Oligomerization Reaction of Ethylene Using Chromium (III) Acetylacetonate

The organic layer sample and polyethylene were obtained in the same manner as in Example 1, except that 10 mg (0.03 mmol) of chromium (III) acetylacetonate was placed instead of 11 mg (0.030 mmol) of tris-tetrahydrofuran chromium trichloride in 10 mL of toluene.

### Comparative Example: Oligomerization Reaction Using Ethylene Oligomerization Catalyst

The organic layer sample and polyethylene were obtained in the same manner as in Example 1, except that TaCl₅ disclosed in U.S. Pat. No. 6,344,594 was used as a catalyst for ethylene oligomerization.

In Examples 1 to 10 and the Comparative Example, the results of preparing 1-hexene or 1-octene were as shown in the following Table 1.

TABLE 1
Catalytic Activity and Analysis Results

| | | | | Selectivity (wt %) | | | |
|---|---|---|---|---|---|---|---|
| | | | Catalytic | | | | Polyethylene |
| | Ligand | Transition metal | activity* | 1-hexene | 1-octene | 1-decene | (PE) |
| Example | Synthesis | Tris-tetrahydrofuran | 1,456 | 21.5 | 77.0 | — | 1.4 |
| 1 | Example 1 | chromium trichloride | | | | | |
| Example | Synthesis | Tris-tetrahydrofuran | 1,213 | 24.2 | 74.1 | — | 1.7 |
| 2 | Example 2 | chromium trichloride | | | | | |
| Example | Synthesis | Tris-tetrahydrofuran | 936 | 19.4 | 78.3 | — | 2.3 |
| 3 | Example 3 | chromium trichloride | | | | | |
| Example | Synthesis | Tris-tetrahydrofuran | 424 | 21.5 | 76.6 | — | 1.9 |
| 4 | Example 4 | chromium trichloride | | | | | |
| Example | Synthesis | Tris-tetrahydrofuran | 562 | 27.2 | 70.1 | — | 2.7 |
| 5 | Example 5 | chromium trichloride | | | | | |
| Example | Synthesis | Tris-tetrahydrofuran | 467 | 32.1 | 65.8 | — | 2.1 |
| 6 | Example 6 | chromium trichloride | | | | | |
| Example | Synthesis | Tris-tetrahydrofuran | 512 | 42.7 | 54.3 | | 3.0 |
| 7 | Example 7 | chromium trichloride | | | | | |
| Example | Synthesis | Tris-tetrahydrofuran | 643 | 39.4 | 57.2 | | 3.4 |
| 8 | Example 8 | chromium trichloride | | | | | |
| Example | Synthesis | Chromium (III) | 1,521 | 22.6 | 76.4 | | 1.0 |
| 9 | Example 1 | 2-ethylhexanoate | | | | | |
| Example | Synthesis | Chromium (III) | 1,468 | 26.5 | 72.5 | | 1.0 |
| 10 | Example 1 | acetylacetonate | | | | | |

| | | | | | |
|---|---|---|---|---|---|
| Comparative Example | 350 | 83.3 | — | 13.5 | 3.2 |

*Catalytic activity unit: g-(1-Hexene + 1-Octene)/mmol-Metal · h

As shown in Table 1, it could be confirmed that when the catalyst compositions of Examples 1 to 10 were used, 1-hexene and 1-octene could be synthesized with high selectivity while exhibiting relatively high catalytic activity, and a more stable polymerization reaction could be carried out than the catalyst of the Comparative Example.

1. A catalyst composition comprising:
an organic ligand compound selected from a group consisting of a compound represented by the following Formula 1 and a compound represented by the following Formula 2; and
a transition metal compound comprising a transition metal of Groups 4 to 12: 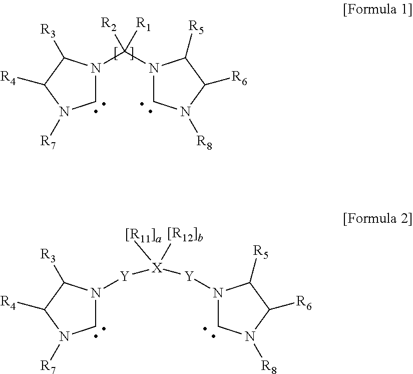
in Formulae 1 and 2,
R₁ to R₈ are optionally the same as or different from each other, and are each independently selected from a group consisting of hydrogen, a straight-chained or branch-chained alkyl having 1 to 10 carbon atoms, a straight-chained or branch-chained alkenyl having 2 to 10 carbon atoms, an aryl having 6 to 20 carbon atoms, an alkylaryl having 7 to 21 carbon atoms, an arylalkyl having 7 to 21 carbon atoms, a cycloalkyl having 3 to 10 carbon atoms, an alkoxy having 1 to 10 carbon atoms, an aryloxy having 6 to 20 carbon atoms, an alkylsilyl having 1 to 10 carbon atoms, an arylsilyl having 6 to 20 carbon atoms, and halogen,
adjacent two or more of R₁ to R₈ are optionally linked to form a ring,
n is an integer of 1 to 20,
X is one element selected from a group consisting of boron (B), carbon (C), nitrogen (N), oxygen (O), silicon (Si), phosphorus (P), and sulfur (S),
a and b are each 0 or 1,
R₁₁ and R₁₂ are optionally the same as or different from each other, and are each independently selected from a group consisting of a straight-chained or branch-chained alkyl having 1 to 10 carbon atoms, a straight-chained or branch-chained alkenyl having 2 to 10 carbon atoms, an aryl having 6 to 20 carbon atoms, an alkylaryl having 7 to 21 carbon atoms, an arylalkyl having 7 to 21 carbon atoms, a cycloalkyl having 3 to 10 carbon atoms, an alkoxy having 1 to 10 carbon atoms, an aryloxy having 6 to 20 carbon atoms, an alkylsilyl having 1 to 10 carbon atoms, an arylsilyl having 6 to 20 carbon atoms, and halogen,
R₁₁ and R₁₂ are optionally linked to each other to form a ring, and
Y and Z are optionally the same as or different from each other, and are each independently selected from a group consisting of a straight-chained or branch-chained alkylene group having 1 to 20 carbon atoms, a straight-chained or branch-chained alkenylene group having 2 to 20 carbon atoms, an arylene group having 6 to 20 carbon atoms, an alkylarylene group having 7 to 20 carbon atoms, and an arylalkylene group having 7 to 20 carbon atoms.

2. The catalyst composition as claimed in claim 1, wherein the catalyst composition is used in a reaction of synthesizing alpha-olefin from ethylene.

3. The catalyst composition as claimed in claim 1, wherein in Formula 1,
R₁, R₂, R₃, R₄, R₅, and R₆ are optionally the same as or different from each other, and are each independently selected from a group consisting of hydrogen, a straight-chained or branch-chained alkyl group having 1 to 10 carbon atoms, a cycloalkyl group having 4 to 8 carbon atoms, and an aryl group having 6 to 20 carbon atoms, and
R₇ and R₈ are optionally the same as or different from each other, and are each independently selected from a group consisting of a straight-chained or branch-chained alkyl group having 1 to 10 carbon atoms, a cycloalkyl group having 4 to 8 carbon atoms, an aryl group having 6 to 20 carbon atoms, an alkylaryl group having 7 to 20 carbon atoms, and an arylalkyl group having 7 to 20 carbon atoms.

4. The catalyst composition as claimed in claim 1, wherein in Formula 2,
R₃, R₄, R₅, and R₆ are optionally the same as or different from each other, and are each independently selected from a group consisting of hydrogen, a straight-chained or branch-chained alkyl group having 1 to 10 carbon atoms, a cycloalkyl group having 4 to 8 carbon atoms, and an aryl group having 6 to 20 carbon atoms,
R₇ and R₈ are optionally the same as or different from each other, and are each independently selected from a group consisting of a straight-chained or branch-chained alkyl group having 1 to 10 carbon atoms, a cycloalkyl group having 4 to 8 carbon atoms, an aryl group having 6 to 20 carbon atoms, an alkylaryl group having 7 to 20 carbon atoms, and an arylalkyl group having 7 to 20 carbon atoms,
X is nitrogen,
Y and Z are optionally the same as or different from each other, and are each an alkylene group having 1 to 5 carbon atoms,
a is 1 and b is 0, and
R₁₁ is selected from a group consisting of a straight-chained or branch-chained alkyl group having 1 to 10 carbon atoms, a cycloalkyl group having 4 to 8 carbon atoms, an aryl group having 6 to 20 carbon atoms, an alkylaryl group having 7 to 20 carbon atoms, and an arylalkyl group having 7 to 20 carbon atoms.

5. The catalyst composition as claimed in claim 1, wherein an unshared electron pair of the compound represented by Formula 1 or the compound represented by Formula 2 and the transition metal of the transition metal compound form a coordination bond.

6. The catalyst composition as claimed in claim 1, wherein the transition metal compound comprises a chromium compound.

7. The catalyst composition as claimed in claim 6, wherein the chromium compound comprises one or more selected from a group consisting of chromium, chromium (III) acetylacetonate, tris-tetrahydrofuran chromium trichloride, and chromium (III) 2-ethylhexanoate.

8. The catalyst composition as claimed in claim 1, wherein the catalyst composition comprises the organic ligand compound in an amount of 0.5 mole to 2.0 mole based on 1 mole of the transition metal compound.

9. The catalyst composition as claimed in claim 1, further comprising:
a co-catalyst.

10. The catalyst composition as claimed in claim 9, wherein the co-catalyst comprises one or more compounds selected from a group consisting of the following Formulae 11 to 13: 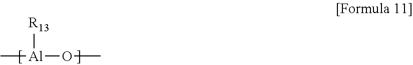
in Formula 11, R₁₃ is an alkyl group having 1 to 10 carbon atoms and r is an integer of 1 to 70, 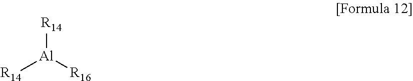
in Formula 12, R₁₄, R₁₅, and R₁₆ are optionally the same as or different from each other, and are each an alkyl group having 1 to 10 carbon atoms, an alkenyl group having 2 to 10 carbon atoms, an alkoxy group having 1 to 10 carbon atoms, or halogen, and at least one of R₁₄, R₁₅, and R₁₆ is an alkyl group having 1 to 10 carbon atoms,
[L-H]⁺[Z(E)₄]⁻or [L]⁺[Z(E)₄]⁻, [Formula 13]
in Formula 11, L is a neutral or cationic Lewis base, [L-H]⁺ or [L]⁺ is a Bronsted acid, and H is a hydrogen atom,
Z is a Group 13 element, and
E's are optionally the same as or different from each other, and are each independently an aryl group having 6 to 20 carbon atoms or an alkyl group having 1 to 20 carbon atoms, wherein the aryl group and the alkyl group of E are each independently are substituted or unsubstituted with one or more functional groups selected from a group consisting of halogen, a hydrocarbyl having 1 to 20 carbon atoms, an alkoxy functional group having 1 to 20 carbon atoms, and a phenoxy functional group.

11. A method for preparing alpha-olefin by a reaction of ethylene with the catalyst composition as claimed in claim 1.

12. The method as claimed in claim 10, wherein a reaction temperature is 0 to 200° C. and a reaction pressure is 1 to 150 bar.
